(19) United States
(12) Patent Application Publication
(10) Pub. No.: US 2021/0177542 A1
(43) Pub. Date: Jun. 17, 2021

(54) POWDER FOR CLEANING AN INTERNAL BODY PART AND/OR AN IMPLANT, METHOD FOR PRODUCING SAID TYPE OF POWDER AND SUITABLE USES

(71) Applicant: FERTON HOLDING S.A., Delémont (CH)

(72) Inventor: Marcel DONNET, Saint Jean de Gonville (FR)

(21) Appl. No.: 16/493,922

(22) PCT Filed: Apr. 3, 2018
(86) PCT No.: PCT/EP2018/058481

§ 371 Date: Feb. 3, 2021

(30) Foreign Application Priority Data
Apr. 3, 2017 (DE) 10 2017 107 124.5

(51) Int. Cl. A61C 3/025; A61K 8/02; B24C 11/00; B24C 7/00

(52) U.S. Cl. CPC A61C 3/025; B24C 7/0046; B24C 11/005; A61K 8/0225

(57) ABSTRACT

A powder for cleaning an internal body part and/or an implant, in particular by means of a powder jet device, wherein the powder is sterile as a result of a sterilizing process.

### TECHNICAL FIELD

The present disclosure relates to a powder for cleaning an internal body part and/or an implant, a method for producing said type of powder and suitable uses.

### BACKGROUND

Cleaning of internal body parts, such as bones and implants, e.g. prior to surgery, is a typical requirement to prophylactically prevent bacterial infections, for example. However, cleaning of implants is challenging, particularly because implants are mostly fabricated of or coated with materials the surfaces of which are susceptible to damage, due to their high biocompatibility requirements and their ability to grow together with human or animal tissue or internal body parts as quickly as possible. Typically, these materials are porous and hydrophilic.

From dental medicine, use of powder-air mixtures is known, which are blasted onto the tooth surface for cleaning purposes by using powder jet devices. However, the use of such a powder-air mixture would require subsequent sterilization of the internal body parts or implants before they can be inserted into the body.

### BRIEF SUMMARY

The disclosure provides a method to be able to clean internal body parts and/or implants as gently as possible without requiring any obligatory post-treatment step.

According to the disclosure, a powder is provided for cleaning an internal body part and/or an implant, in particular by using a powder jet device, wherein the powder is sterile as a result of a sterilizing process, i.e. free from germs and bacteria or essentially free from reproductive microorganisms. Contrary to prior art, a sterile powder according to the disclosure is advantageously provided by which cleaning of internal body parts and/or implants, in particular a surface of the internal body parts and/or implants, may easily and abrasively be performed, even in sterile surgical environments. The powder will advantageously be prepared by the sterilizing process so that no additional post-treatment step, in particular time-consuming sterilization, of the internal body part and/or implant will be required subsequent to cleaning. This advantageously also allows short-term or partial cleaning by employing the powder.

The term implant especially refers to tissue-free implants, e.g. implants for bone or tooth replacement, and the term internal body parts refers to jaw bones or hip bones. For example, the implant is a hip bone implant, a jaw bone implant or equivalents.

The term powder especially refers to any powder or powder mixture that is suitable to be used in a powder jet device, i.e. from which a powder-air mixture can be formed by means of the powder jet device.

In general, the term powder includes any accumulation of particles. This may also be a mixture of particles of different materials or material compositions. Furthermore, the particles of the powder have an advantageous abrasive property, i.e. the particles are designed or selected such that when hitting a surface of the internal body part or implant in a jet of a powder-air mixture, they abrasively clean this surface. Another advantage resides in that the powder may implant—specifically be selected, i.e. the powder may be selected such that probability of surface damage of the respective implant may individually be reduced, when cleaning it using the powder. Preferably, a sterile powder should be understood as one of which the residual content of reproductive microorganisms in the powder is at most 10⁻⁶ colony-forming units, i.e. one million particles of the powder treated in the same way must not contain more than one reproductive microorganism.

Particularly, the term “essentially” means such deviations deviating from the respective exact value by +/−15%, preferably by +/−10%, particularly preferably +/−5% and/or correspond to deviations in the form of changes that are insignificant for proper functioning.

According to another embodiment of the present disclosure, it is provided for the powder to comprise sodium bicarbonate or sodium hydrogen carbonate (NaHCO₂), calcium carbonate (CaCO₂), aluminum trihydroxide (Al[OH]₃), calcium sodium phosphosilicate (Ca—NaO₆PSi), sugar alcohols such as erythritol (C₄H₁₀O₄), disaccharides, especially trehalose (C₁₂H₂₂O₁₁), or isomaltulose and/or glycine or glycocoll, amino acetic acid (C₂F₅NO₂), depending on the application. Suitably, they are substance compositions which are already used in surface treatment of teeth.

It has surprisingly been shown that these compositions are suitable for cleaning bones and/or implants. Particularly preferred are erythritol and/or glycine with average grain sizes of approx. 10-20 μm, preferably approx. 14 μm, which allow comparatively gentle treatment due to their consistency.

In particular, the powder is a powder based on sodium bicarbonate. The powder based on sodium bicarbonate preferably has an average grain size of 12-20 μm. It is preferred for the powder, in particular a powder comprising sodium bicarbonate, to have an average grain size between 5 and 100 μm, preferably between 10 and 50 μm and particularly preferably essentially 17 μm. In particular, it turned out that sodium bicarbonate is relatively easy to sterilize and, furthermore, when reducing it to a grain size of substantially 17 μm, the probability of damage from the powder-air mixture comprising said sodium bicarbonate is generally decreased compared to a coarser-grained sodium bicarbonate powder.

For the numerical grain size values given in this paragraph, the powder is preferably configured such that the grain sizes are distributed around the average grain size with a standard deviation σ being 20 μm, preferably 10 μm, and particularly preferably 5 μm.

Erythritol powder-water mixtures can smoothly be applied to the gingival margin, the pocket entrance and also inside the pocket. Erythritol powder is suitable for the removal of light plaque and stains, for tooth polishing and for biofilm removal, especially on sensitive implant surfaces.

In another embodiment of the present disclosure, it is provided for the powder to have a density between 0.2 and 3 g/cm³, preferably between 0.2 and 2.4 g/ cm³ and particularly preferably between 0.2 and 1.6 g/ cm³. It has advantageously turned out that such powders having a density of between 0.2 and 1.6 g/c cm³ can easily be sterilized and, at the same time, rarely causing surface damage to internal body parts and/or implants when being cleaned with a powder jet device.

It is preferred to use water-based abrasives for powder jet cleaning, although they are generally more difficult to sterilize. Non-water-soluble substances leave a “typically sandy” feeling for the patient, especially in the mouth, and are difficult to removed. This applies both to the oral cavity and to the doctor's office. In addition, there is the risk of damage or retention of non-water-soluble powder particles in the lungs if they cannot be solved and rinsed by using a fluid such as water. Therefore, the water-soluble property is of advantage for the powders according to the disclosure.

In this disclosure, substances are classified as water-soluble if more than 1 g of powder can be added per liter. In another embodiment of the present disclosure, it is therefore provided for the powder to be water soluble, wherein the water solubility at a temperature of 20° C. exceeds 1 g/l, preferably 50 g/l, preferably 100 g/l, or even 100 g/l. The preferred powders have the following properties:

TABLE 1

| | Solubility in water | Density |
|---|---|---|
| Powder | (g/1 at 20° C.) | (g/cm³) |
| Erythritol | 540 | 1.45 |
| Trehalose | 690 | 1.80 |
| Glycine | 225 | 1.61 |
| Sodium bicarbonate | 96 | 2.54 |

Herein, erythritol shows the best values as it has high water solubility compared to very low density, indicating the use thereof in the subgingival area, especially as a sterile powder, with gingiva being open. The disclosure also provides a method for producing a sterile powder, in particular a sterile powder according to the disclosure, comprising the steps of:

providing a powder and

sterilizing the powder in a sterilizing process.

The method according to the disclosure advantageously allows to provide a sterile powder that is suitable for cleaning internal body parts and implants. All the characteristics described for the powders according to the disclosure and advantages thereof may analogously be assigned to the method according to the disclosure and vice versa.

In this context, a sterilizing process especially means a process according to which a powder is prepared which immediately is suitable, i.e. without any further treatment step, as a powder for a powder jet device. For this purpose, with advantage the sterilizing process is to be adapted to the powder such that the powder is sufficiently dry and finely grained after the sterilizing process and is therefore suitable for use in a powder-jet device. Preferably, the moisture in the powder has increased by a maximum of 5%, preferably by a maximum of 2.5% and particularly preferably by 1.5% as a result of the sterilizing process. For example, the moisture may be determined with an infrared hygrometer.

Therefore, the sterilizing process is preferably adapted to or dependent on the powder or type of powder. Preferably, it is provided for the powder to be filled into a powder chamber for a powder jet device immediately subsequently to the sterilizing process. This avoids re-contamination of the sterile powder. If agglomerates have formed as a result of the sterilizing process, the sterile powder may optionally be divided into fine grains again by vortex, sieve, vibration, friction or milling processes.

According to another aspect of the disclosure, the powder should be heated during the sterilization process, especially to a temperature between 110° C. and 210° C., preferably below 180° C., preferably between 120° C. and 160° C., and especially to 160° C.

If a temperature of essentially 160° C. is used, sterilization may advantageously be achieved in a comparatively short time, for example in two hours, without the powder being impaired concerning its ability to form a powder-air mixture. Use of lower temperatures is advantageous in that it allows sterilization of a larger number of powders or powder types without impairing their ability to form the powder-air mixture for a powder jet device.

Expediently it is provided for the powder to be heated between 0.5 and 60 hours, preferably between 1 and 40 hours, and particularly preferably essentially for two hours at temperatures of 100° C. to 250° C., preferably 120° C. to 160° C. If heating is performed for two hours, sufficient sterilization can be achieved comparatively fast; temperatures of around 160° C. are suitable for this purpose. This has an especially positive effect on the manufacturing time and thus also on the costs of the sterile powder.

At lower temperatures, e.g. 120° C. and longer heating, e.g. heating for between 20 and 40 hours, it may be ensured for the powder to be sufficiently sterilized. It is particularly preferred for a powder containing erythritol, trehalose and/or glycine to be heated at 160° C. for two hours. Alternatively, the powder comprising erythritol, trehalose and/or glycine may be heated at 120° C. for 40 hours.

The solubilities or melting points are as follows:

TABLE 2

| | Solubility in water | Melting point |
|---|---|---|
| Powder | (g/1 at 20° C.) | (° C.) |
| Erythritol | 540 | 121 |
| Trehalose | 690 | 215 |
| Glycine | 225 | 233 |
| Sodium bicarbonate | 96 | 851 |

It is shown that the heat sterilization described above is particularly suitable for sodium bicarbonate while the other powders, in particular erythritol, are to be sterilized more appropriately by using other sterilizing methods. As the melting points of those powders and most other powders are not far away from the treatment temperatures, the powder particles may undesirably fuse or adhere to each other. The containers are also required to be temperature-resistant, which results in additional restrictions.

In another embodiment of the present disclosure, it is provided for the powder to be exposed to radiation, in particular β, γ, or X-rays and/or UV-radiation, during the sterilizing process. It is also possible, for example, to bombard the powder with an electron beam. In the case of X-ray radiation, the powder preferably is exposed to the radiation of a radioactive source of radioactive cobalt-60 (⁶⁰CO) or caesium-137 (¹³⁷Cs). For example, for the sterilization of the powder β and/or γ radiation is used. While β radiation is only suitable for surface sterilization due to the low penetration depth thereof, γ radiation has high penetration depth.

Such sterilizing irradiation, e.g. using radioactive beta or gamma radiation, of powder particles, however, results in changes in smell and appearance, which is undesirable.

It is also conceivable that the powder is exposed to vapor, especially water vapor or hydrogen peroxide vapor, during the sterilizing process. For example, the powder may be heated in an autoclave, wherein an interior space of the autoclave is filled with water vapor or hydrogen peroxide vapor, in particular is completely filled with water vapor or hydrogen peroxide vapor. The powder is preferably heated to a temperature of between 60° C. and 132° C. in an autoclave for a period of between 30 min and 60 min. At a duration of 60 min and a temperature of 132° C., a majority of all microorganisms can be destroyed and the powder attains resistance level VI. It has advantageously been proven that calcium carbonate can be sterilized using sterilizing vapor.

However, according to a preferred embodiment of the disclosure, water solubility of the powder particles requires specific restrictions during sterilization, as moistening would cause the powder particles to adhere to each other. Therefore, vapor sterilization is difficult when using water-soluble particles.

According to a preferred embodiment of the present disclosure and in particular for water-soluble and/or heat-sensitive powders, it has surprisingly been found for the powder to be exposed to ethylene oxide (ETO), especially an ethylene oxide gas, during the sterilizing process. Ethylene oxide gas advantageously kills bacteria, viruses and fungi. Sterilization with ETO especially is a low temperature process which is preferably carried out at 5 to 100° C., preferably 2 to 80° C. and particularly preferably 37 to 63° C. It may therefore be used for fumigation of heat-sensitive substances. ETO is preferably used in gaseous form and mixed with other substances such as CO₂ or vapor. This process is advantageously suitable for powders that cannot withstand high temperatures, such as those having low melting points. The use of ETO therefore also permits the use of powders having low melting points. In particular, it is intended that use of ETO should essentially be performed in a vapor-free or vapor-reduced manner, i.e. addition of vapor to the ETO gas will be omitted or the proportion thereof should be reduced in comparison to the proportion used in general. In addition, it is conceivable for the air humidity to be monitored during the sterilizing process. This enables sterilization especially of the light-weighted and highly water-soluble erythritol, wherein erythritol achieves the best results in the subgingival area.

A sterilizing process using ETO has also proven to be particularly advantageous in the case of sodium bicarbonate, in particular sodium bicarbonate having a grain size of essentially 17 μm.

In another embodiment of the present disclosure, it is provided for the powder to be exposed to a sodium hypochlorite (NaCIO) and/or chlorhexidine (CHX) solution. The powder preferably is successively exposed to a sodium hypochlorite (NaCIO) solution and/or a chlorhexidine (CHX) solution, in particular a chlorhexidine (CHX) solution is used chronologically after the sodium hypochlorite solution. This allows the powder to be sterilized. Preferably, a 0.01 to 6 wt % sodium hypochlorite solution, in particular a 0.1 wt % sodium hypochlorite solution, and/or a 0.01 to 2 wt % chlorhexidine (CFIX) solution, in particular a 0.1 wt % solution, is used.

The microorganisms will be transferred into the solution and can be removed together with the used solution, e.g. rinsed out.

It is also conceivable for the powder and the sodium hypochlorite (NaClO) and/or chlorhexidine (CHX) solution to undergo mechanical treatment, such as ultrasonic treatment. For example, in the sterilizing process a process from the document US 2015/0 352 023 A1 is used, the contents of which are explicitly referred to regarding destruction or removal of microorganisms. It is preferable for the powder to be sufficiently dried before use, for example before being filled into a powder chamber.

As vapor is also used in ETO sterilization to increase the effectiveness of the sterilization, restrictions may also be expected for this. To overcome this, the present disclosure advantageously uses a moisture-reduced cycle. With ethylene oxide sterilization (ETO), the treatment usually occurs between 30° C.-60° C., at a relative humidity of over 30%, a gas concentration of 200 mg/l-800 mg/l and takes at least 3 hours.

As the sterile material, i.e. the powder, is sterilized in its packaging, the active substances must be able to act through this packaging. Therefore, the packaging is normally exposed to 100% moisture, which then assumes a value of about 10% to 50%, preferably about 30%, within the packaging, which is a suitable value for an ETO process. For the powders of the present disclosure, a value of 85% moisture (outside the packaging) has proved to be ideal, with a limit of about 90%. The ETO itself does not seem to have a negative effect on the powders tested. As an especially preferred sterilization temperature, 42-52° C., especially 46-48° C. has been established.

The powders of present disclosure are hygroscopic. According to a preferred embodiment, amorphous silica (silicon dioxide) is therefore added to the powders, especially in a volume ratio of 0.1 to 2.5%. This can reduce or even completely prevent absorption of moisture by the powder during sterilizing. The addition of amorphous silica results in hydrophobic coating for the powder particles, thus counteracting the moisture absorption of the powder particles during the sterilizing process. In less hygroscopic powders, addition of amorphous silica may be reduced or even completely omitted. The following powder types may be classified as hygroscopic: sodium hydrogen carbonate, glycine and erythritol. Trehalose is slightly less sensitive.

For the sterilization to be successful, protection of the powder particles from moisture is essential, in regard of maintaining functionality of a water-soluble powder as a blasting agent.

Another approach to obtain a sterile but not hygroscopic powder is to use calcium carbonate as a blasting agent. This powder is not water-soluble and can therefore be preferably sterilized by vapor/heat sterilization.

According to the disclosure, use of a sterile powder according to the disclosure is also provided. All characteristics described for the powder and method according to the disclosure for the production of the sterile powder and advantages thereof may also be transferred analogously to the process of the disclosure and vice versa. For example, the powder-jet device is one as disclosed in WO 2016 142 272 A1 and to the description of which will specifically be referred to concerning the powder-jet device. It is also preferred for the powder to be used to fill interchangeable powder chambers.

### BRIEF DESCRIPTION OF THE DRAWING

Further advantages and characteristics will arise from the following description of preferred embodiments of the disclosure while reference will be made to the accompanying figures. Individual characteristics of the individual embodiments may be combined within the scope of the disclosure.

In the figures:

FIG. 1 is a schematic flow diagram of a process for producing a sterile powder according to an exemplary embodiment of the present disclosure.

### DETAILED DESCRIPTION

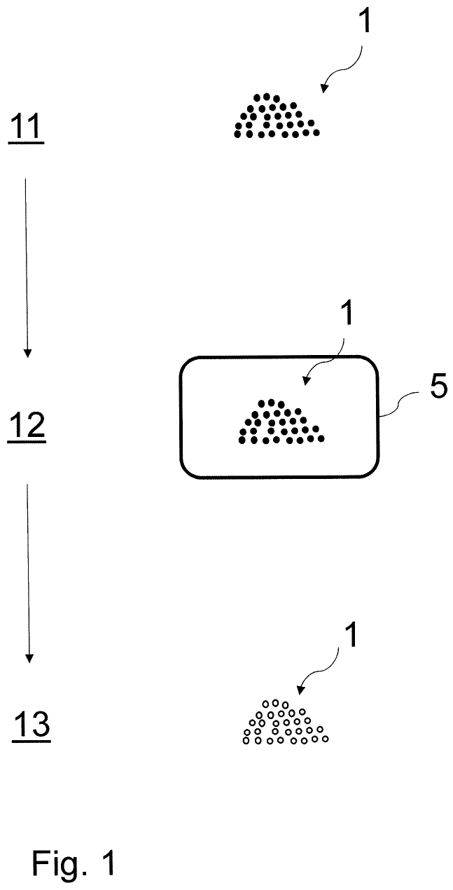

In FIG. 1 a schematic flow diagram of a process for producing a sterile powder 1 according to an exemplary embodiment of the present disclosure is shown. In particular, it is a powder 1 which is for a powder jet device. Typically, a powder-air mixture is produced in such powder jet devices, the powder-air mixture being directed towards an internal body part or implant to be cleaned, for cleaning by the use of the powder jet device, and, for cleaning purposes, the powder 1 in the jet impinging on the internal body part or implant abrasively removes impurities or contaminations, respectively. It is also conceivable for the powder-air mixture to exit the powder jet device together with a fluid, the fluid being mixed with the powder-air mixture and/or enveloping the same. It is advantageous for the powder 1 to be sterilized prior to its use in the powder jet device outside the powder jet device in a sterilizing process 12, i.e. it will be freed from microorganisms such as bacteria and germs. This allows a powder 1 to be advantageously used when cleaning internal body parts or implants, in particular without the need of sterilizing or re-sterilizing the implant or internal body part after cleaning. In particular, the powder 1 is designed such that it does not damage a surface of the implant or the internal body part.

The powder 1 preferably has a density between 0.2 and 3 g/cm³, preferably between 0.2 and 2.4 g/cm³ and especially between 0.2 and 25 1.6 g/cm³. For example, it is a powder 1 comprising sodium bicarbonate, calcium carbonate, erythritol, trehalose and/or glycine, said powder, in particular a powder comprising sodium bicarbonate, having an average grain size between 5 and 100 μm, preferably between 10 and 50 μm and particularly preferably substantially 17 μm. In particular, it has been shown that such powders 1 are also suitable for cleaning implants which, due to their high biocompatibility and ability to rapidly grow in, are susceptible to surface damage because the materials they are made of or coated with are porous and/or hydrophilic.

In the process shown in FIG. 1, a powder 1, in particular sodium bicarbonate having an average grain size of 17 μm, is provided in the beginning. This powder 1 will subsequently be positioned in a closed chamber 5 in which the sterilizing process is performed. For example, in the sterilization process, the powder is heated in chamber 5, exposed to electromagnetic radiation and/or vapor. It has been shown to be particularly advantageous for the powder 1, in particular sodium bicarbonate having an average grain size of 17 μm to be exposed to a gas comprising ethylene oxide. After a residence time of the gas comprising ethylene oxide in the closed chamber 5 has elapsed and after it has been withdrawn from the closed chamber 5, providing 13 the sterile powder 1 occurs. Preferably, the powder 1 is filled into a powder chamber for a powder jet device immediately after the sterilizing process 12, in particular into a sterile powder chamber, to avoid decontamination of the sterile powder 1 immediately after production thereof. For example, the closed chamber 5 comprises an outlet through which the powder 1 can immediately be guided into a powder chamber, which is compatible with a powder jet device, for example.

1. A powder for cleaning an internal body part and/or an implant by means of a powder jet device, wherein the powder is sterile as a result of a sterilizing process.

2. The powder according to claim 1, wherein the powder comprises sodium bicarbonate, calcium carbonate, erythritol, trehalose and/or glycine.

3. The powder according to claim 1, wherein the powder has an average grain size between 5 and 100 μm.

4. The powder according to claim 1, wherein the powder is water soluble, the water solubility at a temperature of 20° C. being above 1 g/l.

5. The powder according to claim 1, wherein the powder further comprises amorphous silica (silicon dioxide) in a volume ratio of 0.1 to 2.5%.

6. A method for producing a sterile powder, comprising the steps of:
providing a powder and
sterilizing the powder in a sterilizing process.

7. The method according to claim 6, wherein the powder is exposed to an ethylene oxide gas in the sterilizing method.

8. The method according to claim 6, wherein the powder is exposed to a sodium hypochlorite (NaClO) and/or a chlorhexidine (CHX) solution.

9. The method according to claim 6, wherein in the sterilizing process the powder is heated to a temperature between 110° C. and 210° C.

10. The method according to claim 6, wherein the powder is exposed to β, γ, X-rays and/or UV-radiation in the sterilizing process.

11. The method according to claim 6, wherein the powder is exposed to a sterilizing vapor in the sterilization process.

12. The sterile powder according to claim 1 configured to be used in a powder jet device for cleaning an internal body part or an implant.
